(19) United States
(12) Patent Application Publication
(10) Pub. No.: US 2020/0072852 A1
(43) Pub. Date: Mar. 5, 2020

(54) IMPROVED METHOD FOR DETECTION OF HELICOBACTER PYLORI -GASTRITIS AND ATROPHIC GASTRITIS WITH RELATED RISKS

(71) Applicant: BIOHIT OYJ, Helsinki (FI)

(72) Inventor: Osmo SUOVANIEMI, Helsinki (FI)

(73) Assignee: BIOHIT OYJ, Helsinki (FI)

(21) Appl. No.: 16/349,742

(22) PCT Filed: Nov. 14, 2016
(86) PCT No.: PCT/FI2016/050800

§ 371 Date: May 14, 2019

(51) Int. Cl. G01N 33/74; G01N 33/569

(52) U.S. Cl. CPC G01N 33/74; G01N 2800/26; G01N 2800/062; G01N 33/56911

(57) ABSTRACT

According to an example aspect of the present invention, there is provided an improved method for screening of asymptomatic and symptomatic subjects with Helicobacter pylori infection, atrophic gastritis with related risks and/or disturbed gastric function based on whole blood samples, sample collection time and sample storage temperature, as well as relevant biomarker content analysis.

### FIELD

The present invention relates to methods for examining a person having the biomarkers indicating Helicobacter pylori infection and/or atrophic gastritis with related risks. More precisely, the present invention relates to methods for improving the analysis steps relating to the screening and detection of the previously mentioned diseases and function of stomach mucosa.

### BACKGROUND

GastroPanel®, the unique Helicobacter pylori test can detect the following conditions:
1) Helicobacter pylori (HP) infection which is an independent risk factor of both gastric cancer and peptic ulcer disease (gastric- and duodenal ulcer).
2) HP infection-induced atrophic gastritis (AG), which in most cases is asymptomatic, as well as the topographic site of AG either in the corpus or in antrum. Apart from HP infection, AG in the corpus with all its clinical sequels can also develop through an autoimmune disease of the gastric mucosa.
2.1) AG of the corpus mucosa leads to low acid output or eventually acid-free (achlorhydric) stomach. This increases the risk of gastric or esophageal cancer, as well as malabsorption of vitamin B12, calcium, magnesium and zinc. In addition, absorption of some medicines, e.g. dipyridamol, some iron preparations and anti-fungal drugs (fluconazol, itraconazol), thyroxin and atazanovir is impaired due to acid free stomach. Calcium deficiency can cause osteoporosis, and vitamin B12 deficiency can contribute to development of Alzheimer's disease, dementia, depression or peripheral neuropathies. Reduced acid output in the stomach can also increase the risk of serious infections in the gastrointestinal- and respiratory tract, including giardiasis, malaria, Clostridium difficile, E. coli EHEC and pneumonia.
2.2) AG of the antrum that increases the risk of gastric cancer. Co-existent AG of the corpus and antrum (pangastritis) is the single most important risk condition for gastric cancer.
3) HP infection also in subjects with AG, MALT-lymphoma or bleeding peptic ulcer, and in those taking PPI medication or antibiotics. In these cases, ¹³C Urea Breath Test (UBT) or stool HP antigen test frequently give false negative results, and HP infection (with all its possible consequences) remains undetected. UBT may give false positive results in subjects with acid-free stomach. In addition, UBT and HP antigen or antibody tests do not detect AG due to HP-infection or autoimmune disease (http://www.biohithealthcare.com/limitations-of-helicobacter-pylori-diagnostics).
4) High acid output of the gastric mucosa, which predisposes to esophageal reflux disease with potential complications. These are ulcerative esophagitis, Barrett's esophagus or lower esophageal cancer.
Symptomatic Helicobacter pylori (HP) infection, atrophic gastritis (AG) and high acid output are indications for gastroscopy.
The GastroPanel® innovation is based on follow-up studies on gastritis patients conducted by research groups in Finland and Estonia and the discovery of the role of Helicobacter pylori in pathogenesis of gastritis and peptic ulcer disease, which led to Nobel Prize in 2005, as well as on Biohit's R&D and the microplate immunoassay analyzers based on the invention of the vertical light beam measurement principle.

More precisely, BIOHIT GastroPanel® is a quantitative ELISA test panel that measures the blood plasma concentration of four biological markers of gastric mucosal structure and function: pepsinogen I (PGI), pepsinogen II (PGII), gastrin-17 (G-17) and Helicobacter pylori IgG antibodies. The main indications of use for GastroPanel® are to aid in the diagnosis of and for screening of asymptomatic and symptomatic subjects with 1) H. pylori infection 2) atrophic gastritis (AG) 3) disturbed gastric function.

GastroPanel® is the first-line diagnostic test for Helicobacter pylori (Hp) infection (5-80% of the world population), for examination of all the patients with dyspepsia (20-40% of the western population), as well as for the screening of atrophic gastritis (AG) with related risks, such as stomach- and oesophageal cancer (1-3). Atrophic gastritis also enhances the risk of malabsorption of vitamin B12, iron, magnesium, zinc, calcium and some medicines. The ¹³C Urea Breath Test (UBT), stool antigen test and antibody tests for Hp infection do not detect atrophic gastritis which is caused by Hp infection or an autoimmune disease nor the level of acid output. In addition, the ¹³C Urea Breath Test and stool antigen test may give false negative results if the patient has a) atrophic gastritis b) MALT lymphoma c) bleeding peptic ulcer disease or d) if the patient is currently receiving antibiotics or PPIs.

GastroPanel® consists of key stomach-specific biomarkers representing the key regulators of normal stomach physiology. These four biomarkers include pepsinogen I (PGI), pepsinogen II (PGII), amidated gastrin-17 (G-17), and Hp-antibodies, designed to give information on both the structure and function of the stomach mucosa (1-6). Most importantly, this panel gives accurate estimates of the capacity of the corpus and antrum mucosa to secrete gastric acid and G-17, respectively, as well as of important gastric pathologies, like inflammation, grade and topography of atrophic gastritis (7-9), which may represent increased risk of gastric cancer and its clinical sequels (1).

Normal plasma levels of all four biomarkers indicate that the stomach mucosa has normal structure and function, whereas the abnormal levels are signs of a non-healthy stomach, reflecting the disturbances in the feedback mechanisms between the acid output of the corpus, PGs and G-17. For G-17 assessment, there are two options; G-17 basal (G-17b) values, and G-17 stimulated (G-17s) values, the latter being particularly important in distinguishing between functional disturbance of the antrum (G-17s normal) and AG in the antrum (G-17s does not increase in AG)(10,11).

Being the first non-invasive diagnostic test for stomach mucosal health, GastroPanel® is unique also in that the results are interpreted by a software application (GastroSoft) (http://www.gastropanel.com), specifically designed for this purpose. GastroPanel® results are classified into one of five possible diagnostic categories: 1) normal mucosa, 2) superficial (Hp) gastritis, 3) AG in the antrum, 4) AG in the corpus, and 5) AG in both antrum and corpus (pangastritis)(11,12). Thus, GastroPanel® is optimized for use together with the Updated Sydney System (USS) for classification of gastritis, which is based on these same 5 diagnostic categories (13).

GastroPanel® has been validated in several large trials based on biopsy-confirmed gastroscopies (14,15), all included in a recent meta-analysis on the subject (14). These studies have been exploited to establish the validated reference (cut-off) values for each individual biomarkers of the panel, using the five histological categories as endpoints. These studies confirm the high accuracy of GastroPanel in detecting the most important study endpoint, moderate-to-severe AG (14-16). Thus, normal values of PGI, PGII and their ratio (PGI/PGII) preclude AG of the corpus with NPV over 95%. In turn, the values of PGI and PGII as well as their ratio below the established cut-off levels predict moderate-to-severe AG with area under ROC curve (AUC) values above 0.950 in adequately powered and USS validated series (1,2,3,16,17).

In brief, the levels of PGI decrease in AG of the corpus (and in pan-gastritis), but remain within the normal range in all other conditions. Elevated PGII levels reflect mucosal inflammation, the highest values being detected in Hp-associated non-AG. The G-17b values are highest in AG of the corpus, because of the missing negative feedback by the acid output from an atrophic corpus, resulting in uninhibited secretion of G-17b by the normal antral mucosa. The same applies to the situation where acid output is inhibited by prolonged use of PPI medication. By definition, when antral mucosa is atrophic and the G cells are depleted, G-17 secretion remains very low even after protein stimulation (G-17s)(17).

Hp IgG antibodies give significant added diagnostic value to the three biomarkers. IgG serology for Hp measures potentially two different conditions: 1) an ongoing Hp-infection, or 2) a previous exposure to Hp. Although GastroPanel® is not capable of making the distinction between these two, the evidence on Hp involvement gives significant added diagnostic value. As the only abnormal marker, Hp implicates an Hp-associated superficial gastritis (with no AG), while associated with abnormalities in the other three markers, elevated Hp-antibody titers confirm the diagnosis of Hp-associated AG (antrum or corpus)(1,3,18,19).

For more details in interpretation of the GastroPanel® results, please refer to http://www.gastropanel.com.

Also, WO 2009/053537 describes the principle methods and concepts relating to the GastroPanel innovation.

Furthermore, Syrjänen (2016) provides systematic review and meta-analysis of all relevant studies on GastroPanel® in diagnosis of atrophic gastritis and corroborates the statement of international experts, advocating GastroPanel® in diagnosis and screening of AG. Due to its high specificity for both AG of the antrum and AG of the corpus, GastroPanel® is truly a test for stomach health.

In the GastroPanel® test concentrations of four biomarkers are determined from a blood sample. These biomarkers: pepsinogen I (PGI), pepsinogen II (PGII) and gastrin-17 (G17), which are secreted by the cells in gastric mucosa, are measured. Additionally, Helicobacter pylori antibodies are measured. An overall interpretation of all four biomarker results provides more reliable and comprehensive understanding of the structure and function of the gastric mucosa than what could be achieved by using the individual biomarkers separately. The concentrations of these biomarkers should however be determined as soon as possible, or to take care that the samples are properly stored and handled before analyses. It is recommended that the blood sample should be centrifuged within 30 minutes from collection of the sample. A special stabilizer substance should be added to the separated plasma tube to prevent degradation of any of the analytes. The stabilizer enables the storage of the sample at room temperature (20-25° C.) for three days, or in the refrigerator (2-8° C.) for up to seven days. In case of longer delay before analysis, the sample tolerates storage at −20° C. for up to 2 weeks. For any longer storage time, the sample should be kept at −70° C. However, if it is not possible to centrifuge the sample it is not possible to use the stabilizer as the red blood cells become haemolysed. Therefore, the effect of temperature and time during storage and transportation of the whole blood sample is of high interest.

### SUMMARY OF THE INVENTION

The invention is defined by the features of the independent claims. Some specific embodiments are defined in the dependent claims.

According to a first aspect of the present invention, there is provided a method for screening of asymptomatic and symptomatic subjects with Helicobacter pylori infection, atrophic gastritis with related risks and/or disturbed gastric function, based on obtaining a whole blood sample, recording of sample collection time, or sample storage temperature, or combination of both, measuring biomarker concentration of interest, and determining an initial biomarker concentration and status of stomach mucosa based on such information.

According to a second aspect of the present invention, there is provided a device or a program which determines the initial biomarker concentration based on measurement of the plasma or serum biomarker concentration, time between whole blood sample collection and plasma or serum sample analysis, and storage temperature of the whole blood sample.

According to a third aspect of the present invention, the initial concentration of a biomarker, such as gastrin-17, determined by such means is used in a non-invasive test for screening of asymptomatic and symptomatic subjects with Helicobacter pylori infection, atrophic gastritis with related risks and/or disturbed gastric function.

The present invention is based on the finding that the concentration of for example gastrin-17 biomarker drops monotonically versus time and temperature. If the concentration is higher than the limit of quantification and the information of the storage temperature and time of sample collection is known, the initial gastrin-17 concentration can be reliably estimated and used for interpreting the results of stomach mucosa health.

These and other aspects, together with the advantages thereof over known solutions are achieved by the present invention, as hereinafter described and claimed.

More precisely, the method of the present invention is characterized by what is stated in claims 1, 2 and 3.

A device or a program according to the present invention is characterized by what is stated in claim 7, and the use according to the present invention by what is stated in claim 9.

Considerable advantages are obtained by means of the invention. For example, blood samples do not require immediate centrifugation for separation of plasma or serum for further analysis. Instead, samples can be collected, stored and sent for further analysis as whole blood samples without processing them first. In the analysis step where the biomarkers are then measured, the time of sample collection and storage temperature are used to interpret and correct the obtained results, which may have been affected by for example protein degradation. This makes the actual clinical work less time-depending and time-consuming, and also saves costs. For example, it is not required to do the analysis of each sample on-site where the blood sample is taken, but just to collect samples to be processed and analyzed later on. Also internal logistics in the research unit are thus improved. In addition, blood samples do not require stabilizers for storage purposes, but instead can be stored at for example room temperature as whole blood samples before further analytical use.

Next, the present technology will be described more closely with reference to certain embodiments.

### EMBODIMENTS

The present technology relates to the effect of temperature and time during storage and transportation of whole blood samples, when examining a person having or assuming to have the GP biomarkers indicating a Helicobacter pylori infection and/or atrophic gastritis with related risks.

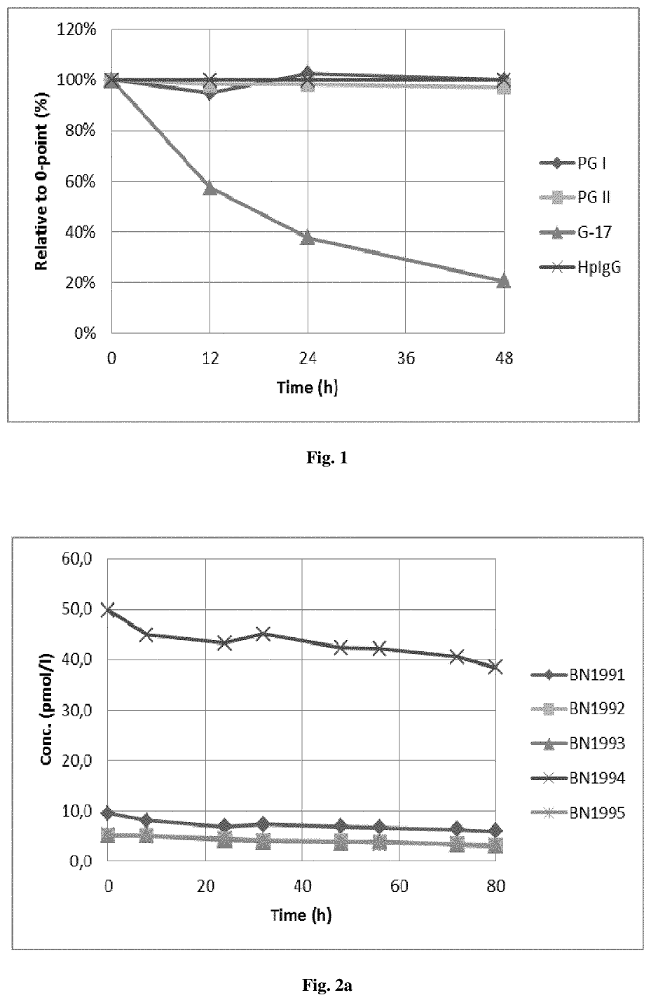

It has been shown that pepsinogen I, pepsinogen II and Helicobacter pylori stands well without substantial degradation for few days storage at normal room temperature, unlike gastrin-17, as illustrated in FIG. 1. In this context different protein degradation events like oxidation, photodegradation, disulfide scrambling, deamidation, aggregation, precipitation, dissociation, fragmentation etc., are not of interest, but simply the effect of storage time and temperature.

FIG. 1 is a diagram showing relative concentration change of pepsinogens and gastrin-17, when samples are stored as whole blood at 21° C. with different period of time before analyses.

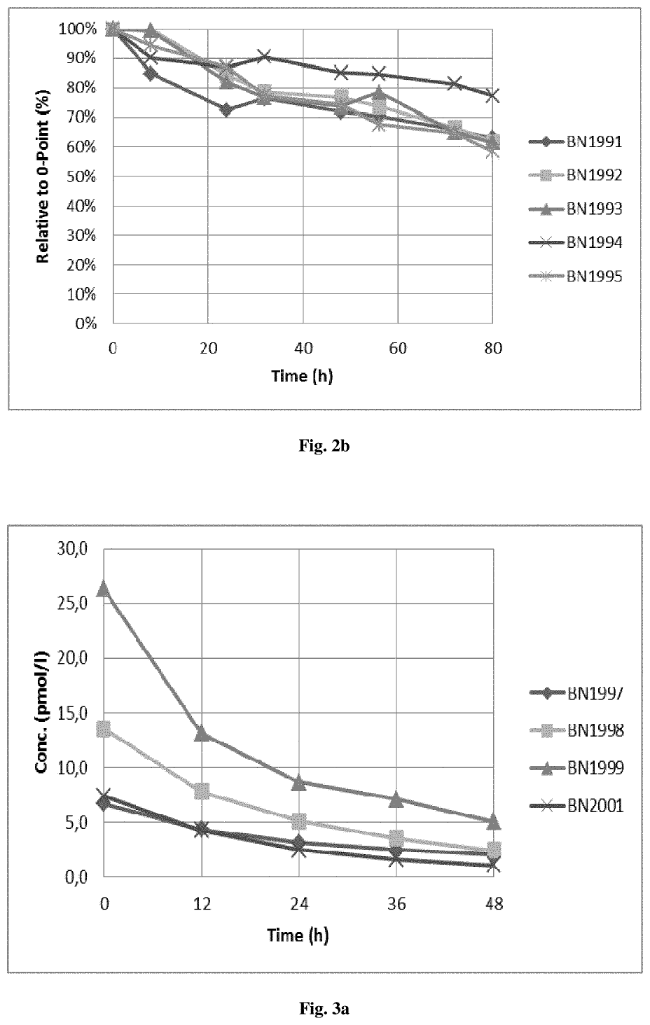

FIGS. 2 to 4 illustrate gastrin-17 concentration after storing the samples for 12, 24, 36 and 48 hours at 4° C., room temperature of 21° C., and 30° C. The relative drop of concentration is about 20%, 40% and 80% within 24 hours at 4° C., 21° C. and 30° C., respectively.

More precisely, FIG. 2a is a diagram showing the gastrin-17 concentration of samples stored as whole blood at 4° C. with different period of time before analysis

FIG. 2b is a diagram showing the gastrin-17 relative concentration change of samples as whole blood at 4° C. with different period of time before analysis.

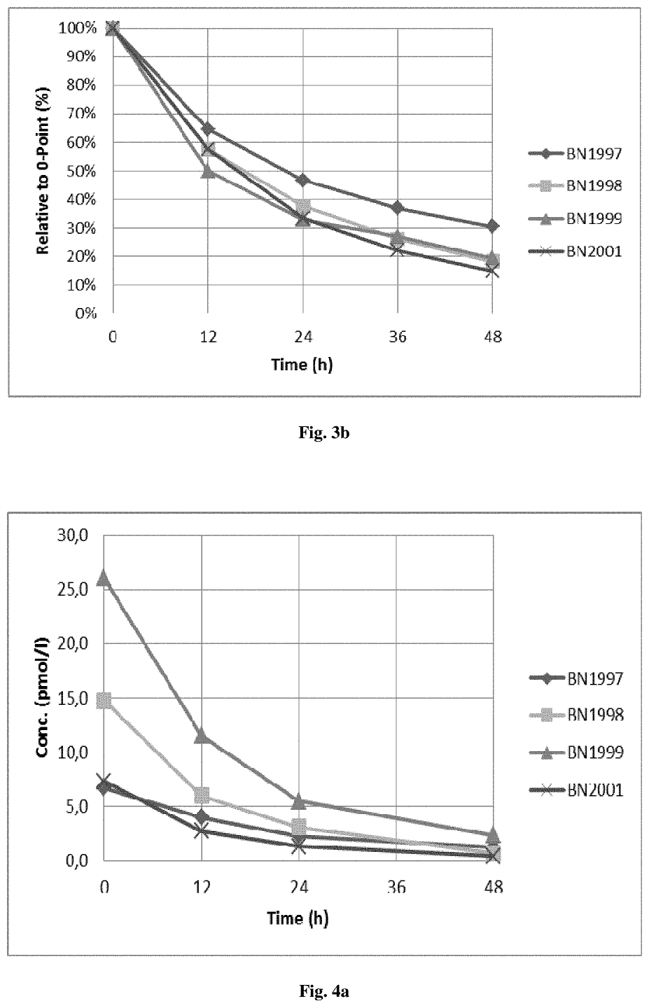

FIG. 3a is a diagram showing the gastrin-17 concentration of samples as whole blood at 21° C. with different period of time before analysis.

FIG. 3b is a diagram showing the gastrin-17 relative concentration change of samples as whole blood at 21° C. with different period of time before analysis.

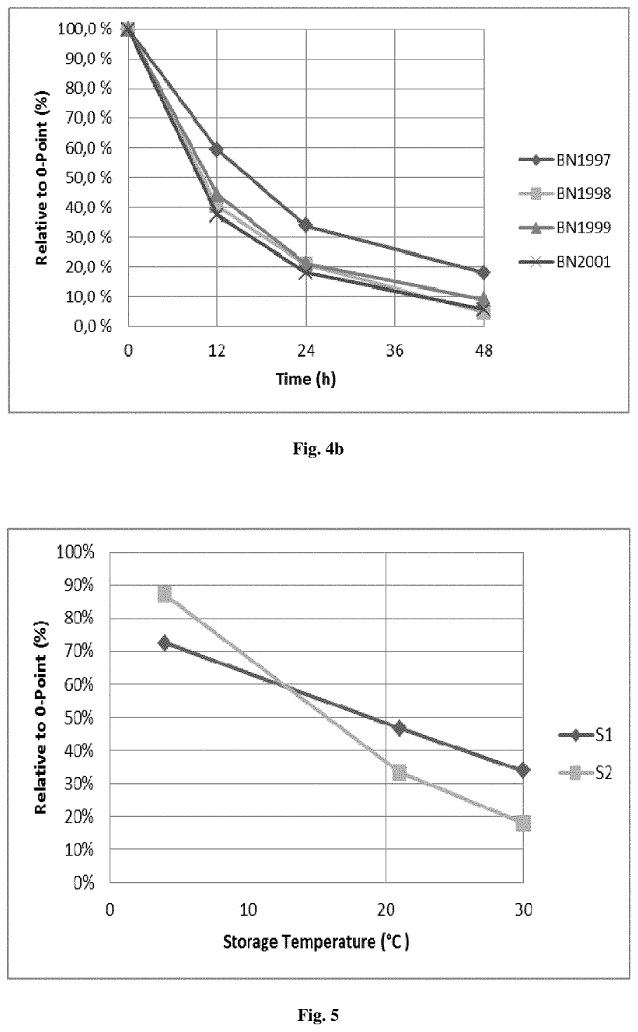

FIG. 4a is a diagram showing the gastrin-17 concentration of samples as whole blood at 30° C. with different period of time before analysis.

FIG. 4b is a diagram showing the gastrin-17 relative concentration change of samples as whole blood at 30° C. with different period of time before analysis.

Storage/transportation temperature has significant effect on the gastrin-17 degradation rate as shown in FIG. 5. Thus, FIG. 5 is a diagram showing one day (24 h) storage of whole blood in different temperatures for two different samples.

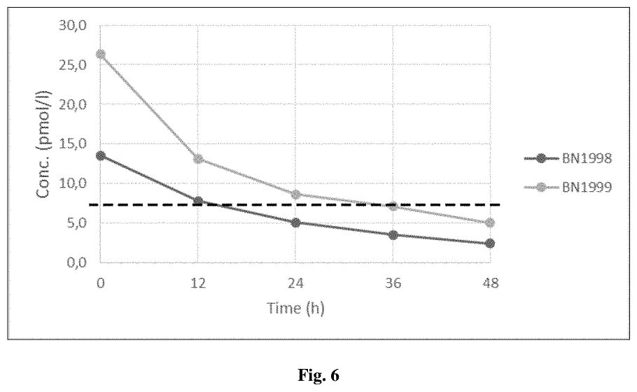

FIG. 6 is a diagram showing gastrin-17 concentration of samples stored as whole blood at 30° C. with different period of time before analyses. The dashed line presents gastrin-17 high reference of 7 pmol/l (an example of high reference; any higher concentration indicates low acid).

One embodiment of the present invention is a method for screening of asymptomatic and symptomatic subjects with Helicobacter pylori infection, atrophic gastritis with related risks and/or disturbed gastric function, said method including the steps of:
a) obtaining a whole blood sample from a subject,
b) recording the time of the whole blood sample collection,
c) quantitatively measuring gastrin-17 concentration of a plasma or serum sample separated from the whole blood sample,
d) determining initial gastrin-17 concentration by comparing the obtained values from steps b) and c) to reference values, and
e) interpreting the structure and/or function of the stomach mucosa based on said comparison.

Another embodiment of the present invention is a method for screening of asymptomatic and symptomatic subjects with Helicobacter pylori infection, atrophic gastritis with related risks and/or disturbed gastric function, said method including the steps of:
a) obtaining a whole blood sample from a subject,
b) recording the storage temperature of the whole blood sample collection,
c) quantitatively measuring gastrin-17 concentration of a plasma or serum sample separated from the whole blood sample,
d) determining initial gastrin-17 concentration by comparing the obtained values from steps b) and c) to reference values, and
e) interpreting the structure and/or function of the stomach mucosa based on said comparison

Yet another embodiment of the present invention is a method for screening of asymptomatic and symptomatic subjects with Helicobacter pylori infection, atrophic gastritis with related risks and/or disturbed gastric function, said method including the steps of:
a) obtaining a whole blood sample from a subject,
b) recording the time of the whole blood sample collection,
c) recording the storage temperature of the whole blood sample collection,
d) quantitatively measuring gastrin-17 concentration of a plasma or serum sample separated from the whole blood sample,
e) determining initial gastrin-17 concentration by comparing the obtained values from steps b), c) and d) to reference values, and
f) interpreting the structure and/or function of the stomach mucosa based on said comparison.

A method, wherein in addition to gastrin-17 concentration also concentrations of pepsinogen I, pepsinogen II and Helicobacter pylori IgG antibodies are determined from the plasma or serum sample, belongs to the scope of the present invention.

One suitable, but not limited to, analytical function for estimating the initial concentration of the samples is as follows. If assumed that the concentration decrease is proportional to the number of molecules

$$- \frac{dN}{dt} \approx N , or - \frac{dN}{N} = kdt ,$$

where
N is the number of molecules, k indicates rate at which decrease will take place, and t is the time.
   The solution to the 1^{st} order differential equation can be presented as follows:

N(t)=N₀e^{-kt}, where
N₀ is the initial number of molecules i.e. initial concentration of the analyte, and N(t) is the measured number if molecules (≈concentration).
   Because the measured concentration is affected by the storage temperature, the function can be presented by general form:

N(t, T)=N₀e^{-kt}f(T), where
T is the storage temperature of the sample.
   The concentration half time t_{1/2} can be solved from the formula:

$$k ( T ) = \frac{{t}_{\frac{1}{2}}}{ln ( 2 )} f ( T )$$

The half time

$${t}_{\frac{1}{2}}$$

and k(T) can be estimated from the measured data.

As the concentration drops monotonically vs time and temperature, and if the concentration is higher than the Limit of Quantification (LoQ), and information of the storage temperature and time of sample collection is known, it is possible to estimate the initial concentration and use that information when interpreting the results.

According to one embodiment of the present invention, high (vs the reference range) gastrin-17 concentration indicates acid-free stomach. If a sample that has been stored for some time before measurement still gives high concentration value, also the initial concentration must have been high. Such a situation is illustrated by the sample BN1999 in the FIG. 5, assuming that measurement have been carried out next day (24 hours) from sample collection. The interpretation on stored sample results is as valid as if the sample would have been analyzed right after collection.

Further, if it is sure that the temperature chain has not exceeded certain temperature, it can predict concentration vs. reference range, and enable the use of that when interpreting the results. For example, when sample BN1998 in FIG. 5 has been stored at room temperature (21° C.) before measurement, and the measured value is 5 pmol/l, it can be assumed that the half time is less than 24 hours, and therefore be confident that the initial value has been higher than the example high reference of 7 pmol/l. Thus, also this case indicates lack of acid in the stomach and e.g. with low pepsinogen level will increase the specificity of atrophic gastritis diagnosis.

Furthermore, if it is assumed that the sample has been stored for 24 hours before measurement, and the value is about 2 pmol/l, it indicates that the acid content of the stomach is normal, less than the upper limit of the reference range.

However, if the measured concentration is below the LoQ, it is not possible to predict the initial concentration. In that case the gastrin-17 results cannot be used for the interpretation. The GastroPanel® interpretation must then be made based on the other biomarker values.

According to one embodiment the whole blood sample is collected and stored without stabilizers.

According to one embodiment the time period between whole blood sample collection and further analysis from the plasma or serum sample is between 0 to 120 hours, more preferably between 0 to 72 hours and most suitably between 0 to 48 hours.

According to one embodiment the whole blood sample is collected and stored at temperatures between 0 to 30° C., for example at 4° C., 21° C. or 30° C., before the separation of plasma and/or serum and analysis of the biomarker concentrations.

A device or a program which determines an initial biomarker concentration based on measurement of plasma or serum biomarker concentration, time between whole blood sample collection and plasma and/or serum sample analysis, and storage temperature of the whole blood sample belongs to the scope of the present invention.

Preferably, the plasma or serum biomarker concentration is above the limit of quantification, the time between whole blood sample collection and plasma and/or sample analysis is between 0 to 48 hours, and the storage temperature of the whole blood sample is between 0 to 30° C.

Thus, such device or program may be for example GastroSoft program, which is designed for interpreting GastroPanel results, which has been modified to take into consideration the above mentioned essential parameters of the present invention.

One further embodiment of the present invention is to use the methods as disclosed in a non-invasive test for screening of asymptomatic and symptomatic subjects with Helicobacter pylori infection, atrophic gastritis with related risks and/or disturbed gastric function.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

At least some embodiments of the present invention find industrial application for example in screening and examination of patients with dyspepsia, as well as for the screening and detection of atrophic gastritis (AG) with related risks, such as stomach- and oesophageal cancer.

### CITATION LIST

### Patent Literature

1. WO 2009/053537

### Non Patent Literature

1. Agréus L, Kuipers E J, Kupcinskas L, Malfertheiner P, Di Mario F, Leja M, Mahachai V, Yaron N, van Oijen M, Perez Perez G, Rugge M, Ronkainen J, Salaspuro M, Sipponen P, Sugano K and Sung J: Rationale in diagnosis and screening of atrophic gastritis with stomach-specific plasma biomarkers. Scand J Gastroenterol 2012; 47:136-147.
2. Storskrubb T, Aro P, Ronkainen J, Sipponen P, Nyhlin H and Talley N J: Serum biomarkers provide an accurate method for diagnosis of atrophic gastritis in a general population: the Kalixanda study. Scand J Gastroenterol 2008; 43:448-1455.
3. Wikström M: Assessment of stomach health by “chemical gastroscopy”. Eur Gastroenterol Rev 2012; 1-6.
4. Lomba-Viana R, Dinis-Ribeiro M, Fonseca F, Vieira A S, Bento M J and Lomba-Viana H: Serum pepsinogen test for early detection of gastric cancer in a European country. Eur J Gastroenterol Hepatol 2012; 24:37-41.
5. Miki K: Gastric cancer screening using the serum pepsinogen test method. Gastric Cancer 2006; 9:245-253.
6. Bornschein J, Selgrad M, Wex T, Kuester D and Malfertheiner P: Serological assessment of gastric mucosal atrophy in gastric cancer. BMC Gastroenterol 201212:10. doi: 10.1186/1471-230X-12-10.
7. Germaná B, Di Mario F, Cavallaro L G, Moussa A M, Lecis P, Liatoupolou S, Comparato G, Carloni C, Bertiato G, Battiestel M, Papa N, Aragona G, Cavestro G M, Iori V, Merli R, Bertolini S, Caruana P and Franzé A: Clinical usefulness of serum pepsinogens I and II, gastrin-17 and anti-Helicobacter pylori antibodies in the management of dyspeptic patients in primary care. Dig Liver Dis 2005; 37:501-508.
8. Miki K, Ichinose M, Shimizu A, Huang S C, Oka H, Furihata C, Matsushima T and Takahashi K: Serum pepsinogens as a screening test of extensive chronic gastritis. Gastroenterol Jpn 1987; 22:133-141.
9. Samloff I M, Varis K, Ihamaki T, Siurala M and Rotter J I: Relationships among serum pepsinogen I, serum pepsinogen II, and gastric mucosal histology. A study in relatives of patients with pernicious anemia. Gastroenterol 1982:83:204-209.
10. Korstanje A, den Hartog G, Biemond I and Lamers C B: The serological gastric biopsy: a non-endoscopical diagnostic approach in management of the dyspeptic patient: significance for primary care based on a survey of the literature. Scand J Gastroenterol 2002 236 (Suppl): 22-26.
11. Oksanen A, Sipponen P, Miettinen A, Sarna S and Rautelin H: Evaluation of blood tests to normal gastric mucosa. Scand J Gastroenterol 2000; 35:791-795.
12. Varis K, Sipponen P, Laxen F, Samloff I M, Huttunen J K, Taylor P R, and The Helsinki Gastritis Study Group: Implications of serum pepsinogen I in early endoscopic diagnosis of gastric cancer and dysplasia. Scand J Gastroenterol 2000:35:950-956.
13. Dixon M F, Genta R M, Yardley J H and Correa P: Classification and grading of gastritis. The updated Sydney System. International Workshop on the Histopathology of Gastritis, Houston 1994. Am J Surg Pathol 1996; 20:1161-1181.
14. Väänänen H, Vauhkonen M, Helske T, et al. Non-endoscopic diagnosis of atrophic gastritis with a blood test. Correlation between gastric histology and serum levels of gastrin-17 and pepsinogen I: a multicenter study. Eur J Gastroenterol Hepatol 2003:15:885-891.
15. Telaranta-Keerie A, Kara R, Paloheimo L, Härkönen M and Sipponen P: Prevalence of undiagnosed advanced atrophic corpus gastritis in Finland: an observational study among 4,256 volunteers without specific complaints. Scand J Gastroenterol 2010; 45:1036-1041.
16. Dinis-Ribeiro M, Yamaki G, Miki K, Costa-Pereira A, Matsukawa M and Kurihara M: Meta-analysis on the validity of pepsinogen test for gastric carcinoma, dysplasia or chronic atrophic gastritis screening. J Med Screen 2004; 11:141-147.
17. Sipponen P, Ranta P, Helske T, Kääriäinen I, Mäki T and Linnala A. Serum levels of amidated gastrin-17 and pepsinogen I in atrophic gastritis: an observational case-control study. Scand J Gastroenterol 2002; 37:785-791.
18. Benberin V, Bektayeva R, Karabayeva R ym. Prevalence of H. pylori infection and atrophic gastritis among ymptomatic and dyspeptic adults in Kazakhstan. A hospital-based screening with a panel of serum biomarkers. Anticancer Res 2013; 33:4595-4602.
19. Syrjänen K J, Sipponen P, Härkönen M, Peetsalu A, Korpela S. Accuracy of GastroPanel testing in detection of atrophic gastritis. Eur J Gastroenterol Hepatol 2015; 27:102-104.
20. Misiewicz J J. The Sydney System: a new classification of gastritis. Introduction. J Gastroenterol Hepatol. 1991; 6:207-208.
21. Sipponen P, Price A B. The Sydney System for classification of gastritis 20 years ago. J Gastroenterol Hepatol. 2011; 26: Suppl 1:31-34.
22. Malfertheiner P, Megraud F, O'Morain C A, Atherton J, Axon A T, Bazzoli F, Gensini G F, Gisbert J P, Graham D Y, Rokkas T, El-Omar E M, Kuipers E J. European Helicobacter Study Group. Management of Helicobacter pylori infection—the Maastricht IV/Florence Consensus Report. Gut 2012; 61:646-664.
23. Syrjänen K. A Panel Of Serum Biomarkers (GastroPanel®) in Non-invasive Diagnosis of Atrophic Gastritis. Systematic Review and Meta-analysis. Anticancer Research 36: 5133-5144 (2016).

1. A method for screening of asymptomatic and symptomatic subjects with Helicobacter pylori infection, atrophic gastritis with related risks and/or disturbed gastric function, said method including the steps of:
a) obtaining a whole blood sample from a subject,
b) recording the time of the whole blood sample collection,
c) quantitatively measuring gastrin-17 concentration of a plasma or serum sample separated from the whole blood sample,
d) determining initial gastrin-17 concentration by comparing the obtained values from steps b) and c) to reference values, and
e) interpreting the structure and/or function of the stomach mucosa based on said comparison.

2. A method for screening of asymptomatic and symptomatic subjects with Helicobacter pylori infection, atrophic gastritis with related risks and/or disturbed gastric function, said method including the steps of:
a) obtaining a whole blood sample from a subject,
b) recording the storage temperature of the whole blood sample collection,
c) quantitatively measuring gastrin-17 concentration of a plasma or serum sample separated from the whole blood sample,
d) determining initial gastrin-17 concentration by comparing the obtained values from steps b) and c) to reference values, and
e) interpreting the structure and/or function of the stomach mucosa based on said comparison

3. A method for screening of asymptomatic and symptomatic subjects with Helicobacter pylori infection, atrophic gastritis with related risks and/or disturbed gastric function, said method including the steps of:
a) obtaining a whole blood sample from a subject,
b) recording the time of the whole blood sample collection,
c) recording the storage temperature of the whole blood sample collection,
d) quantitatively measuring gastrin-17 concentration of a plasma or serum sample separated from the whole blood sample,
e) determining initial gastrin-17 concentration by comparing the obtained values from steps b), c) and d) to reference values, and
f) interpreting the structure and/or function of the stomach mucosa based on said comparison.

4. The method according to claim 1, wherein in addition to gastrin-17 concentration also concentrations of pepsinogen I, pepsinogen II and Helicobacter pylori IgG antibodies are measured from the plasma or serum sample.

5. The method according to claim 1, wherein the whole blood sample is collected and stored without stabilizers.

6. The method according to claim 1, wherein the whole blood sample is collected and stored at temperatures between 0 to 30° C. before the separation of plasma and/or serum and analysis of the biomarker concentrations.

7. A device or a program, which determines an initial biomarker concentration based on
measurement of plasma or serum biomarker concentration,
time between whole blood sample collection and plasma or serum sample analysis, and
storage temperature of the whole blood sample.

8. The device or a program according to claim 7, wherein
the plasma or serum biomarker concentration is above the limit of quantification,
the time between whole blood sample collection and plasma or serum sample analysis is between 0 to 48 hours, and
the storage temperature of the whole blood sample is between 0 to 30° C.

9. The method according to claim 1 for use in a non-invasive test for screening of asymptomatic and symptomatic subjects with Helicobacter pylori infection, atrophic gastritis with related risks and/or disturbed gastric function.
